(19) United States
(12) Patent Application Publication
(10) Pub. No.: US 2002/0193313 A1
(43) Pub. Date: Dec. 19, 2002

(54) Method of increasing the efficacy of chemotherapy in the treatment of tumors

(76) Inventor: Kourosh Bagheri, Redondo Beach, CA (US)

Correspondence Address: LYON & LYON LLP, 633 WEST FIFTH STREET, SUITE 4700, LOS ANGELES, CA, 90071, US

(21) Appl. No.: 09/883,731

(22) Filed: Jun. 18, 2001

(51) Int. Cl. A61K 31/70

(52) U.S. Cl. 514/23

(57) ABSTRACT

A method for increasing the efficacy of chemotherapy in the treatment of cancer. Two nutritional solutions are provided to the tumor in an alternating manner. The first nutritional solution is formulated to inhibit tumor growth and the second nutritional solution is formulated to stimulate tumor growth. The tumor is denied all other external sources of nutrition. When an elevated population of the tumor cells are determined to be synchronously in the S phase of the cell cycle, chemotherapy treatment is begun. In an alternative embodiment, tumors in vitro are monitored to identify which nutrients are preferentially used by the tumor cells. The first and second solutions are then formulated based on the nutrients so identified.

### BACKGROUND OF THE INVENTION

1. Field of the Invention

The field of the present invention is the treatment of cancer cells using therapy techniques such as chemotherapy and radiation therapy.

2. Background

Nutrition has long been recognized as an important factor in the treatment of malignant disease. Warren, S. (1932) Am. J. Med. Sci.: 184:610, and, more recently, others (DeWys, W. D., Begg. C., Lavin, P. T., et al. (1980) Am. J. Med. Sc.: 69:491-497) have shown that malnutrition can significantly influence the mortality and shorten the survival time of patients with disseminated disease. Furthermore, in a patient with a compromised nutritional status, the treating physician may elect to delay cancer therapy for fear of reducing the patient's oral intake of nutrients because of nausea. In 1967, Ducrick, et al. (1968) Surgery, 64: (1969) 134, Ann. Surg., 169:974, introduced a safe and effective method to parenterally feed patients until gastrointestinal function could be restored. Soon, total parenteral nutrition (TPN) was used for patients in many diseased states such as congenital anomalies of the gastrointestinal tract, trauma, inflammatory bowel disease, and liver disease. Fischer, U.S. Pat. No. 3,950,529.

Copeland and Dudrich (1976) Curr. Probl. Cancer, 1:3, recognized the potential of parenteral feeding techniques to solve nutritional problems in a cancer population. It was then theorized that if nutritional therapy in conjunction with cancer therapy could improve host nutritional status, then host survival should improve. Also, preliminary uncontrolled studies showed that host tolerance and tumor response to anti-neoplastic therapy improved when malnourished patients received TPN, advancing the theory that the force feeding of nutrients reduced host toxicity and sensitized tumors to chemotherapeutic agents. Isell, B. F., Valdivieso, M., Zaren, H. A., et al. (1978) Cancer Treat Rep., 62:1139; Lanzotti, V. J., Copeland, E. M. III, George, S. L., et al. (1075) Cancer Treat Rep., 59:437.

In the 1970's, several prospective randomized trials were conducted to test these theories. These trials involved patients requiring systemic chemotherapy for testicular carcinoma, small cell lung carcinoma, lymphoma, lung adeno carcinoma, and colorectal carcinoma. Lanzotti, et al., (1075) Cancer Treat. Rep., 59:437; Samuels, M. L., et al. (1981) Cancer Treat. Rep., 65:615-627; Popp, M. D., et al., (1981) Cancer Treat. Rep., 65:129-135; Valdivieso, M., et al., (1981) Cancer Treat Rep., 659 (suppl. 5):145; Nixon, D. W., et al., (1981) Cancer Treat. Rep., 659 (suppl. 5):121-128; Jordan, W. M., et al., (1987) Cancer Treat. Rep., 65:197. The results of these trials showed that TPN did not improve host tolerance to chemotherapy. Furthermore, tumor response rates were independent of host nutritional status. In fact, one report suggested that TPN may accelerate disease progression and hasten patient demise. Nixon, D. W., et al., (1981) Cancer Treat. Rep., 65 (suppl. 5):121-128.

Several studies in animal-tumor models in the late 1970's and early in the 1980's showed that TPN can result in tumor growth and in some cases, accelerate growth. Cameron, I. L., (1981) Cancer Treat. Rep., 65 (suppl. 5):93; Buzby, G. P., Mullen, J. L., Stein, T. P., et al., (1980) Cancer, 45:2940. For some tumors, tumor growth can also occur during host starvation, indicating that tumors are able to obtain the exogenous or endogenous nutrients they require to satisfy their energy needs and fuel their biosynthetic pathways even in nutritionally compromised patients and even if they hasten the patient's demise. Sauer, L. A., Nagel, W. O., Dauchy, R. T., et al., (1986) Cancer Res., 46:3469. Therefore use of a standard TPN formulation was contraindicated for patients with malignant disease.

Later studies have examined the relationship between tumor growth and specific nutritional constituents. For example, increased tumor growth was observed in one study in which rats were given a TPN solution deficient in leucine but high in other branched chain amino acids. Torosian, M. H., Stein, T. P., Presti, M. E., (1983) Proc. of the Fed'n of Am. Societies for Experimental Bio., 16:5987. However, in another study, tumor growth was suppressed in rats that were given a TPN solution having no methionine. Goseki, N., Yamazaki, S., Endo, M., Onodera, T., Kosaki, G., Hibino, Y., Kuwahata, T., (1992) Cancer, 69(7):1865-72. Specifically formulated TPN solutions have also been shown to inhibit tumor growth. In particular, formulated TPN solutions containing specified quantities of amino acids, as disclosed in U.S. Pat. Nos. 4,998,724 and 5,189,025, and containing structured lipids, as disclosed in U.S. Pat. No. 5,081,105, have been found to inhibit tumor growth.

Achieving optimal response to chemotherapy is hindered by various host and tumor specific metabolic and micro-environmental factors. Poor tumor blood flow, abnormal pH, hypoxia, cellular drug uptake and efflux mechanisms, specific inherent and/or acquired resistance mechanisms, cellular heterogeneity, and patient immune and nutritional compromise are among the most commonly recognized mechanisms accounting for treatment failure in cancer. Moll, M., Vaupel, P. (eds.): Springer-Verlag Berlin Heidelberg, New York, 1998.

One roadblock to effectively treating a tumor with chemotherapy is that many chemotherapeutic agents damage cells only during specific phases of the cell cycle. All eukaryotic cells progress through several phases of dormancy, active synthesis and replication during their life spans. These phases are: (1) G0 phase, the dormant, non-replicating, phase where most non-neoplastic cells spend the majority of their life spans performing useful functions for the body; (2) G1 phase, the first gap phase that is characterized by intense protein synthesis as the cell prepares for DNA synthesis and eventual replication; (3) S phase, the DNA synthetic phase that is characterized by duplication of nuclear DNA; (4) G2 phase, the second gap phase of protein synthesis wherein cytoskeletal and membrane structures are prepared for duplication; and (5) M phase, or mytosis, wherein the cell physically separates into two cells, the two cells typically being in the G0 phase. The duration of the cycle as well as the number of times each cell goes through the cycle prior to apoptosis varies with cell type. This holds true for both normal cells and cancer cells. Most cancer cells, however, differ from normal cells in that they do not respond to normal cellular replication signals and thus are continuously in the replication part of the cell cycle without having long periods of dormancy in between. Therefore, the chemotherapeutic agents typically used are selected to target replicating cells, and most commonly those cells in the S phase of the replication process, thus making chemotherapy more effective in treating the tumor.

However, in targeting cells in the S phase of the replication process, the chemotherapeutic agents also harm normal cells that are in the S phase. Among normal cells, bone marrow progenitors, intestinal lining and hair cells have the highest proportion of their population undergoing replication at any one time. As such, these cells normally are the most likely to exhibit side effects from chemotherapy treatment. The maximum tolerable level of side effects on these cells often defines the maximum dosage and the duration of chemotherapy treatment. Treatment methods are therefore being developed which maximize the effects of chemotherapy on cancer cells in a tumor while simultaneously minimizing the effects on normal cells.

In developing such methods, the cellular composition and the microenvironment of a tumor needs to be taken into consideration. Within any given solid tumor there are several populations of cells that are at different phases of the cell cycle. Many cells in a tumor are rapidly proliferating and as such are highly responsive to chemotherapy. Other cells are irreversibly arrested and destined to die with or without therapy. The most important fraction of cells, in terms of those which are the most difficult to treat with chemotherapy, are either reversibly arrested or slowly cycling. Such cells are difficult to treat because they are either arrested in a phase of the cell cycle outside the replication phase or they are cycling slowly enough that the chemotherapeutic agents are either metabolized or cleared before these cells enter into the replication phase. In addition, these cells may reside closer to the center of the tumor mass where they are protected by the abnormal microenvironment of the tumor, thus making them even more inaccessible to the chemotherapeutic agents. Moll, M. , Vaupel, P. (eds.): Springer-Verlag Berlin Heidelberg, New York, 1998. The challenge in improving the efficacy of chemotherapy therefore lies in stimulating the growth of these groups of arrested and slowly cycling cells just before the administration of chemotherapy without stimulating the proliferation of normal cells.

Despite cancer cells' lack of sensitivity to normal growth signals, cancer cell growth, and thus tumor growth, can be manipulated. Studies have been performed which correlate the stimulation of tumor growth with the effectiveness of chemotherapy. See J. Nat'l Cancer Inst. 56:597-601, 1976; and Schwartz, G. F., Bendon, M. L., Graham W. P. III, Blakemore, W. S., (1971) Am. J. Surg., 121:169-173. Growth stimulation in these and other similar studies have been achieved through the administration of TPN solutions. For example, studies have shown reduced tumor growth in mice implanted with adenocarcinoma on a casein reduced diet (Jose, D. G., Good, R. A., (1973) J. Exp. Med., 137:1-9), in mice implanted with adenocarcinoma on a diet deficient in phenylalanine, valine, and isoleucine (Theurer, R. C., (1959) Arch. Biochem. Biophys., 81:448-455), and in rats implanted with Yoshida Sarcoma and given a methionine TPN solution (Goseki, N., Yamazaki, S., Endo, M., Onodera, T., Kosaki, G., Hibino, Y., Kuwahata, T., (1992) Cancer, 69(7):1865-72). Conversely, another study has shown increased tumor growth in laboratory rats implanted with adenocarcinoma following the administration of a TPN solution deficient in leucine but high in other branched chain amino acids. Torosian, M. H., Stein, T. P., Presti, M. E., (1983) Proc. of the Fed'n of Am. Societies for Experimental Bio., 16:5987.

Additional studies have shown that inducing tumor growth increases the population of cancer cells that are in the S phase of replication. The increased S phase cell population has been achieved by administering a standard TPN solution to rats implanted with mammary adenocarcinoma (Torosian, M. H., Mullen, J. L., Miller, E. E., Zinsser, K. R., Stein, T. P., Buzby, G. P., (1983) J. Parenteral and Enteral Nutrition, 7(4):337-345; and Torosian, M. H., Mullen, J. L., Miller, E. E., Wagner, K. M., Stein, T. P., Buzby, G. P., (1983) Surgery 94:291-299) and hepatoma (Cameron, J. L., (1981) Cancer Treat. Rep., 65(suppl):93-99). In one clinical trial, patients with gastric carcinoma were given a methionine and cystine depleted TPN solution. Using the cycle specific agents mitomycin and 5-flourouracil, these patients showed an improved response to chemotherapy compared to patients given the standard TPN solution. Goseki, N., Maruyama, M., Nagai, K., Kando, F., Endo, M., Shimoju, K., Wada, Y., (1995) Gan To Kagaku Ryoho, 22(8):1028-35. Such studies show that the cell cycle in different types of cancer may be manipulated through nutritional stimuli to improve response to chemotherapy. However, these studies also show that the stimuli are specific to the type of cancer and that the effectiveness is often dependent upon the microenvironment of the tumor and the complex relationship between the tumor and the host.

Increased tumor response during the S phase of the cell cycle has given rise to a strategy called S phase synchronization, which is used to render a greater number of cancer cells in a tumor susceptible to the effects of chemotherapy. In general, S phase synchronization has been achieved in vitro by introducing stressors to induce cell phase arrest followed by removal of the stressor, thus resuming replication. Although this method is useful for inducing S phase synchronization in vitro, the stressors, such as supratherapeutic doses of drugs or extreme hypothermia, could not be used in an in vivo setting because of their toxicity.

Inducing S phase synchronization on a tumor in vivo, however, has been shown to be possible by other means, some of which have shown less toxicity than others. For example, in one clinical study hormone regulation was used to induce S phase synchronization in patients having locally advanced breast cancer. These patients were first given Tamoxifen to induce cell phase arrest, and subsequently given estrogen to stimulate cell growth. Tumors in approximately 82% of the patients who underwent the synchronization treatment showed favorable response to phase specific chemotherapy, as opposed to only 43% of those without synchronization. Sjovall, M. P., Malmstrom, P., (1997) Acta Oncologica, 36(2):207-12. In a second clinical trial of patients in the advanced stages of different types of cancer, topotecan was given to induce S phase synchronization and was followed by the administration of doxorubicin. The treatment, however, resulted in increased hematologic toxicity due to an increase in the number of hematologic progenitor cells entering into the S phase. Tolcher, A. W., O'Shaughnessy, J. A., Weiss, R. B., Zujewski, J., Myhand, R., Schneider, E., Hakim, F., Gress, R., Goldspiel, B., Noone, M. H., Brewster, I., Gossard, M. R., Cowan, K. H., (May 1997) Clin. Cancer Res., 3(5):755-60.

Other studies, conducted in vitro, have brought to light additional stimuli that may be used to induce S phase synchronization. Methods that have been used to arrest the cell cycle include administering peplomycin in various types of cancer (Tsuboi, T., (April 1985) Nippon Sanka Fuinka Gakkai Zasshi, 37(4):629-36), depriving TSH dependent thyroid carcinoma cells of TSH (Degrassi, A., Monoco, M. C., Lisignoli, G., Belvedere, O., Toneguzzi, S., Malangone, W., Bonora, M. L., Piacentini, A., Lavaroni, S., Scarbolo, M., Ambesi-Impiobato, F. S., Fachini, A., (December 1998) J. Exp. Clin. Cancer Res., 17(4):527-32) and hypothermia combined with radiation treatment of Guerin's tumor (Balmukhanov, S. V., Karakulov, R. K., (1978) Neoplasma, 25(5):585-93). Other stimuli have been found to arrest the cell cycle in a specific phase. Such cycle specific stimuli are reflected in reports, including the following: leucine deprivation led to the arrest of in vitro colon cancer in the G0 phase (Hwang, H. S., Davis, T. W., Houghton, J. A., Kinsella, T. J., (January 2000) Cancer Res., 60(1):92-100); quinidine led to the arrest of in vitro breast cancer in the G1 phase (Wang, S., Melkoumian, Z., Woodfork, K. A., Cather, C., Davidson, A. G., Wonderlin, W. F., Strobl, J. S., (September 1998) J. Cell Physiol., 176(3):456-64); epidermal growth factor led to the arrest of in vitro breast cancer in the G1 phase (Dong, X. F., Berthois, Y., Dussert, C., Isnardon, D., Palmari, J., Martin, P. M., (November-December 1992) Anticancer Res., 12(6B):2085); glucocorticoids blocked in vitro rat hepatoma cells from entering the G1 phase (Sanchez, I., Goya, L., Vallerga, A. K., Firestone, G. L., (March 1993) Cell Growth Differ., 4(3):215-25); and for in vitro T-cell lymphoma, mimosine led to arrest in the G1 phase, hydrea led to arrest in the S phase, nocodazole led to arrest in the M-phase, and aphidicoline led to arrest late in the G1 phase (Yerly-Motta, V., Pavy, J. J., Herve, P., (1999) Biotech Histochem., 74(3):119-28). In each of the cited studies, no matter in which phase the cell cycle was arrested, the arrest led to a later S phase synchronization and therefore increased the efficacy of chemotherapy treatment. Thus, S phase synchronization currently provides the most promising route to treating cancer effectively using chemotherapy.

In view of the foregoing, it becomes evident that no single stimulus achieves S phase synchronization among all types of cancer under all environmental conditions. Therefore, a method is needed for determining which stimuli are best suited for achieving S phase synchronization in any given population of cancer cells and administering such stimuli to achieve S phase synchronization, and therefore optimal results from chemotherapy treatment.

### SUMMARY OF THE INVENTION

The present invention is directed to a method for improving the efficacy of chemotherapy in the treatment of cancer. In a first separate aspect of the present invention, two nutritional solutions are provided to a tumor in an alternating manner while depriving the tumor of other external sources of nutrition. The first nutritional solution is formulated to inhibit tumor growth and the second is formulated to stimulate tumor growth. The tumor is provided with the nutritional solutions in this alternating manner until the population of tumor cells that is synchronously in the S phase of the cell cycle is elevated. Once such synchronization in the S phase is achieved, chemotherapy treatment is initiated.

In a second separate aspect of the present invention, the nutritional components which are used preferentially by the tumor cells are identified, and these nutritional components are used to formulate the first and second nutritional solutions.

In a third separate aspect of the present invention, the nutritional components comprise nutrients from the groups of amino acids, lipids, electrolytes, and trace minerals.

In a fourth separate aspect of the present invention, the first nutritional solution is depleted of the nutritional components used preferentially by the tumor.

In a fifth separate aspect of the present invention, the first nutritional solution is enhanced with nutrients which compete for cellular uptake with the nutritional components used preferentially by the tumor.

In a sixth separate aspect of the present invention, the second nutritional solution is enhanced with the nutritional components used preferentially by the tumor.

In a seventh separate aspect of the present invention, the second nutritional solution is depleted of nutrients which compete for cellular uptake with the nutritional components used preferentially by the tumor.

In an eighth separate aspect of the present invention, the second nutritional solution is enhanced with carbohydrates to further stimulate the entry of tumor cells into the S phase of the cell cycle.

In a ninth separate aspect of the present invention, cellular absorption of radioactive labeled molecules is used to determine when the proportion of tumor cells in the S phase is elevated.

In a tenth separate aspect of the present invention, the first and second nutritional solutions are delivered to the tumor using Total Parenteral Nutrition.

In an eleventh separate aspect of the present invention, any of the foregoing aspects may be employed in combination.

Accordingly, it is an object of the present invention to provide a method of increasing the efficacy of chemotherapy in the treatment of cancer. Other objects and advantages will appear hereinafter.

### DETAILED DESCRIPTION OF THE PREFERRED EMBODIMENTS

The present invention is directed toward a method of increasing the efficacy of chemotherapy in the treatment of cancerous tumors. The method comprises first providing the cancer cells with a first nutritional solution which is specifically formulated to inhibit tumor growth. This first nutritional solution may be any one of the presently known TPN solutions which have been shown to inhibit tumor growth, such as the solutions disclosed in U.S. Pat. No. 4,998,724, U.S. Pat. No. 5,081,105, and U.S. Pat. No. 5,189,025, the disclosures of which are incorporated herein by reference. The first solution may also be formulated according to the specific nutritional requirements of the tumor in accordance with the process set forth below. In inhibiting tumor growth through the administration of the first nutritional solution, a population of the cancer cells within the tumor will be reversibly arrested in the cell cycle due to a lack of nutrition.

Following the administration of the first nutritional solution, a second nutritional solution is provided to the cancer cells. This second nutritional solution is formulated to stimulate tumor growth. Therefore, the second nutritional solution may be any standard TPN solution which is known in the art to stimulate tumor growth, or it may be formulated based on the specific nutritional requirements of the tumor in accordance with the process set forth below. In providing the stimulatory solution to tumor cells which have been reversibly arrested, many of those tumor cells will be induced into the replication part of the cell cycle.

Administration of the first and second nutritional solutions is continued in an alternating manner until a determination is made that the population of cancer cells that are synchronously in the S phase is elevated. Once such a determination is made, chemotherapy treatment is begun using S phase specific chemotherapeutic agents, such as cytosine arabinoside (ARA-C), 5-fluorouracil (5-FU), and methotrexate (MTX). However, in order to provide the greatest increase in the efficacy of chemotherapy, administration of the nutritional solutions is continued until a determination is made that more than 75% of the cancer cells, and more preferably greater than 90% of the cancer cells, are synchronously in the S phase.

When performing the method of the invention on a tumor in vitro, S phase synchronization can be evaluated by known methods, including flow cytometry or thymidine labeling. Flow cytometry has the advantage of providing rapid evaluations and being less labor intensive. Therefore, flow cytometry is the preferred method where large quantities of in vitro tumor cultures must be assessed. Thymidine labeling, on the other hand, has the advantage of being able to more reliably verify the portion of tumor cells in the S phase. Thymidine labeling, therefore, is preferably used for small quantities of tumor cultures or as a final verification step for tumor cultures when cytometry is used. Each of these methods for measuring tumor growth are well known in the art and are therefore only briefly described herein. See Denekamp, J., Kallman, R. F., (1973) Cell Tissue Kinet, 6:217-227.

In performing flow cytometry, cells are sampled from the tumor and trypsinized using 0.2% trypsin and 0.05% EDTA in phosphate-buffered saline (PBS), centrifuged, and washed twice with PBS. The cells are then re-suspended in 1 ml of PBS, fixed with 5 ml of 70% ethanol solution, and stored at 4° C. overnight. At the time of analysis, cells are collected by centrifugation at 8000 G and suspended in 0.2 mg/ml of propidium iodide in Hank's balanced salt solution (HBS) containing 0.6% NP-40. RNase at 1 mg/ml is added and the mixture is incubated in the dark at room temperature for 30 minutes. After filtration, the cells are run through a cytometer, such as the EPICS 753 flow cytometer which is manufactured by Coulter of Hileaheah, Fla. The percentage of cells in each phase of the cell cycle is then calculated. This can most conveniently be done using software, such as the multi-cycle software version 2.53 which is a product of Phoenix Flow Systems of San Diego, Calif.

In performing thymidine labeling, tumor cultures are labeled by adding to the culture 0.5 μCi of 3H-thymidine at a specific activity of 18,500 mCi/mmol for one hour and then washing the culture with ice cold PBS. Cells are extracted from the culture by placing the culture in a 10% cold trichloro-acetic acid (TCA) for 15 minutes on ice and then suspended in 0.5 N NaOH. The TCA-insoluble fraction of cells is extracted by centrifugation at 1000G and the radioactivity of this portion is determined by liquid scintillation counting. The results are expressed in terms of the percentage of cells in the S phase just prior to the thymidine labeling step.

In vivo, the determination of S phase synchronization cannot be easily made using cytometry or thymidine labeling, as both require sampling the tissue several times to determine the percentage of cells in the S phase. In addition, cytometry and thymidine labeling would involve an undesirable delay from the time a sample is taken to the time chemotherapy treatment could begin. Therefore, more direct and immediate methods are desired for measuring the degree of S phase synchronization of an in vivo tumor.

Presently, the best process for measuring in vivo S phase synchronization is through positron emission tomography (PET). PET scanning is an established method for determining the proliferative activity of cells in vivo and is currently the most direct process available for verifying in vivo S phase synchronization. The utility of PET scanning in detecting tissues with a high S phase portion of cells has been demonstrated in various animal and human studies of cancer. See Mankoff, D. A., Dehdashti, F., Shields, A. F., (2000) Neoplasia, 2(1-2):71-88; Boni, R., Blauestein, P., Dummer, R., von Schulthess, G. K., Schubiger, P. A., Steinert, H. C., (1999) Melanoma Res., 9(6):569-573; Ryser, J. E., Blauenstein, P., Remy, N., Weinreich, R., Hasler, P. H., Novak-Hofer, I., Schubiger, P. A., (1999) Nuc. Med. & Bio., 26(6):673-679; and Bergstrom, M., Lu, L., Fasth, K. J., Wu, F., Bergstrom-Pettermann, E., Tolmachev, V., Hedberg, E., Chemg, A., Langstrom, B., (1998) J. Nuc. Med., 39(7):127-79, the disclosures of which are incorporated herein by reference. For example, PET scanning has been effective in demonstrating lower proliferative activity of colorectal cancer post radiation. Schiepers, C., Haustermans, K., Geboes, K., Filez, L., Bormans, G., Penninckx, F., (1999) Amer. Cancer Soc. 85(4):803-811. A direct correlation has also been established between activity seen in PET scans and tumor proliferation rates, both in vitro and in vivo. Ryser, J. E., Blauenstein, P., Remy, N., Weinreich, R., Hasler, P. H., Novak-Hofer, I., Schubiger, P. A., (1999) Nuc. Med. & Bio., 26(6):673-679. PET scanning may therefore be used to determine prognosis in cancer patients and to search for the presence of residual tumor foci. Bergstrom, M., Lu, L., Fasth, K. J., Wu, F., Bergstrom-Pettermann, E., Tolmachev, V., Hedberg, E., Chemg, A., Langstrom, B.; (1998) J. Nuc. Med., 39(7):1273-79.

In using the process of PET scanning with the method of this invention, following the serial administration of the first and second nutritional solutions, a tracer that has been verified as an in vivo proliferation marker, such as bromine-76-bromodeoxyuridine or 3H-thymidine, is introduced to the in vivo environment. The proliferation marker is permitted sufficient time to be distributed via the normal in vivo circulatory functions prior to performing the PET scan. The results of the PET scan are then interpreted for proliferative activity in the vicinity of the tumor. The actual results of the PET scan and the reading thereof are well known to those skilled in the art and are therefore not included in detail herein.

It is expected that during the course of practicing this invention additional means of determining S phase synchronization for in vivo tumors will be revealed. For example, one potential means includes verification that the timing for S phase synchronization for tumors in vitro is the same as for those in vivo. Another potential means which may prove reliable is the correlation of markers in fluids containing drainage from the tumor. For example, the microenvironment of a tumor is highly acidic when the tumor is rapidly replicating due to the production of lactic acid as a byproduct of the replication process. Therefore, higher than normal levels of lactic acid in fluids containing drainage might act as a marker for the replication activity of a tumor, such fluids including direct fluid drainage from the tumor or venous fluids, such as blood, into which the drainage flows. The levels of other potential markers in the fluids, such as oxygen, glucose, and nucleotides, might also prove useful in detecting the replication activity of a tumor.

One embodiment of the invention includes determining the nutritional needs of a tumor by examining the specific components of fluids draining from the tumor. The specific components examined may include amino acids, lipids, electrolytes, and trace metals. Further studies of the nutritional needs of particular types of tumors may reveal other components, also nutritionally required by the tumors, for which the fluids should be examined.

The prior art has established that the levels of amino acids provided to a tumor through TPN can inhibit or induce tumor growth. In addition, the sera of cancer patients have been observed to have differential levels of certain amino acids as compared to controls. See Clarke, E. F., Lewis, A. M., Waterhouse, C., (1978) Cancer, 42:2909-13; Waterhouse, C., Mason, J., (1981) Cancer, 48:939-44; Ye, S. L., (June 1989) Chung Hua I Hsueh Tsa Chih, 69(6):319-20; and Brenner, U., Schindler, J., Muller, J. M., Walter, M., Keller, H. W., (October 1985) Infusionsther Klin Ernahr, 12(5):241-5. That the changes in amino acid levels can be attributed to the tumor is supported by the detection of elevated rates of uptake of some amino acids in cancer cells in culture. See Cendan, J. C., Souba, W. W., Copeland III, E. M., Lind, D. S., (May 1995) Ann. Surg. Oncol., 2(3):257-65; and Cendan, J. C., Souba, W. W., Copeland III, E. M., Lind, D. S., (September 1996) Ann. Surg. Oncol., 3(5):501-8. It has also been demonstrated that even in the face of host starvation tumors can increase the efficiency of their uptake mechanisms to extract amino acids from the serum in order to sustain their own growth. Copeland, E. M., Mac Fadyen, B. V., Lanzotti, V. J., Dudrick, S. J., (February 1975) Am. J. Surg., 129:167-173. Furthermore, significantly higher total amino acid levels have been measured in biopsies of hepatocellular, colon and gastric tumors when compared to normal controls as well as non-cancerous tissues of the same patients. Watanabe, A., Toshihiro, H., Tatsuro, S., Hideo, N., (1984) Cancer, 54:1875-82. Specific elevations of certain amino acids, such as methionine, phenylalanine, tyrosine and lysine, have also been observed in tumor tissues. Palwik, T. T., Souba, W. W., Sweeney, T. J., Bode, B. P., (2000) J. Surg. Res., 91(1):15-25.

While the exact nature of a tumor's dependence on amino acids is unknown, such observations have led to a hypothesis that genetic alterations in cancer cells lead to changes in their energy producing mechanisms and their nutrient uptake and utilization characteristics. While it is possible that specific mutations cause cancer cells to become completely dependent on the uptake of certain amino acids from serum, it is more likely that tumor cells do not completely lose the ability to synthesize these amino acids. Instead, the intracellular synthetic mechanisms likely become relatively inefficient at synthesizing these amino acids, thus making serum uptake the preferred method for obtaining them. However, regardless of the reason, some amino acids that are non-essential for normal cells appear to become essential for tumor cells, with the result being the reliance on uptake of certain amino acids from serum. This is further supported by the fact that TPN solutions specially formulated with amino acids can inhibit tumor growth.

However, generalizations about the specific components that become essential to tumor growth are likely to be inaccurate due to tumor heterogeneity. Furthermore the nutritional dependencies are likely to be dynamic in nature and dependent upon factors such as the microenvironment of the tumor and its specific genetic abnormalities. As these factors change, so might the tumor cells' specific metabolic requirements. In addition, tumors have shown the ability to grow regardless of host starvation.

In determining the nutritional needs of a tumor, therefore, a full analysis of the levels of amino acids present is appropriate because of the critical role amino acids appear to play in the growth of a tumor. Once the amino acid needs of a tumor are identified, the amino acid content of the stimulatory and inhibitory nutritional solutions may be formulated. In formulating the stimulatory nutritional solution, the only likely requirement is that it contain sufficient amounts of the amino acids the cancer cells use preferentially during replication. However, depending on the tumor being treated, it may be desirable to significantly enhance the quantities of the preferential amino acid in the stimulatory solution. Additionally, under some circumstances it may be advantageous to deplete the stimulatory solution of nutrients which compete for cellular uptake with the preferred nutritional components, thereby enhancing the uptake of the preferred components. Other requirements of the stimulatory solution may be discovered through the practice of this invention.

The inhibitory solution may be formulated so that it is wholly depleted of the amino acids identified as being preferentially used by the tumor. Alternatively, the inhibitory solution may contain reduced to negligible amounts of the preferential amino acids. However, where the inhibitory solution contains at least some amount of the preferential amino acids, care should be taken to not destroy the inhibitory nature of the nutritional solution. A third alternative which may be used in formulating the inhibitory solution is to provide the solution with additional amounts of amino acids which compete for uptake with the preferential amino acids. In this manner, the tumor cells can still be deprived of the essential amino acids.

Lipids and their metabolic byproducts play integral roles in tumor growth and response to chemotherapy. Higher levels of alpha linoleic acid uptake have been observed in rapidly growing tumors. Demetrokopoulos, G. E., Brennan, M. F., (February 1982) Cancer Res., 42(supp):756a-765a. The uptake rate of lipids has been observed to be lower under conditions of slow replication and hypothermia. See Dauchy, R. T., Blask, D. E., Sauer, L. A., Brainard, G. C., Krause, J. A., (Oct. 1, 1999) Cancer Lett., 144(2):131-6; and Ding, X. U., Iverson, P., Cluck, M. W., Knezetic, J. A., Adrian, T. E., (Jul. 22, 1999) Biochem. Biophys. Res. Comm., 261(1):218-23. Therefore, lipid membrane constituents appear necessary for cell replication and appear to be needed in abundance for tumor cells to replicate quickly.

Consistent with such observations is the fact that alpha linoleic acid has been found to stimulate tumor growth. See Demetrokopoulos, G. E., Brennan, M. F., (February 1982) Cancer Res., 42(suppl.):756a-765a; and Burns, C. P., Spector, A. A., (June 1990) Nutr. Rev., 48(6):233-240. This fatty acid potentiates the effects of chemotherapy presumably by increasing cellular turnover and metabolism and by possibly increasing the number of cells in the S phase. McCarthy, M. F., (February 1996) Med. Hypotheses, 46(2):107-15. In addition, lipids potentiate the cytotoxic action of radiation and chemotherapy through the generation of free radicals via peroxidation. Cancer cells are more prone to the effects of these free radicals since many of them lack the appropriate free radical scavenging enzymes.

Therefore, in formulating the lipid content of the inhibitory nutritional solution, it is likely desirous to eliminate lipids from the formulation because of their effects on tumor growth. However, it is possible that for different types of malignancies and depending on the circumstances, it may not be useful to eliminate all lipids from the inhibitory nutritional solution, but such determinations must be made on a case-by-case basis and should be based on experience gained from practicing the invention on similar types of in vitro tumors. In formulating the stimulatory nutritional solution, it is likely desirous to include lipids. However, the exact amount of lipids included in the stimulatory nutritional solution must also be made on a case-by-case basis.

Concentrations of biologically active cations, namely magnesium, potassium and calcium, and the trace metals iron and zinc have been shown to alter cancer cell growth and metabolism. Magnesium depletion was found to inhibit cancer cell growth but not normal cell growth in vitro. Sgambato, A., Wolf, F. I., Faraglia, B., Cittadini, A., (August 1999) J. Cel. Physiol., 180(2):245-54. This effect occurred with a lag time of 16 hours post depletion. Upon repletion of magnesium there was a 24 hours lag time prior to resumption of S phase. Additionally, magnesium and calcium depletion was found to increase adriamycin uptake and potentiate its cytotoxicity in vitro. Park, S. M., Han, S. B., Hong, D. H., Lee, C. W., Park, S. H., Jeon, Y. J., Kim, H. M., (February 2000) Arch. Pharm. Res., 23(1):59-65. This effect was attributed to alterations in cell membrane fluidity in the presence of these electrolytes.

Potassium uptake by cells has also been implicated in the growth regulatory mechanisms of cancer and normal cells. Potassium depletion led to a 30-60% growth inhibition in sarcoma and hepatoma cells in mice. See Ching, N., Grossi, C., Jham, G., Zurawinsky, H., Ching, C. Y., Nealon, Jr., T. F., (June 1984) Surgery, 95(6):730-8; and Young, G. A., Parson, F. M., (1977) Europ. J. Cancer, 13:103-113. In the same study, magnesium depletion led to a 40% growth inhibition while the combination of potassium and magnesium depletion led to a 45-80% growth inhibition. Furthermore, the effects of quinidine on cycle progression of breast cancer cells has been studied, with the conclusion that membrane potentials, as determined by relative sodium and potassium concentrations, were affected by quinidine and were crucial in regulating the cell cycle progression in the cells studied. Wang, S., Zaroui, M., Woodfork, K. A., Davidson, A. G., Wonderlin, W. F., Strobl, J. S., (1998) J. Cell. Phys., 176:456-464.

In formulating the inhibitory nutritional solution, it is unlikely that severe deficiencies in any of the above electrolytes can be induced in vivo settings without causing significant cardiovascular side effects. However, these studies underscore the need for sufficient quantities of these electrolytes to be present in the stimulatory solution for the growth phase of the therapy.

It is, however, difficult to determine the effects of pH on tumor growth and response to chemotherapy. This is partly due to the lactate buildup that accompanies increased metabolism and lowers the pH in the immediate environment of the tumor. A lower pH can also impede the uptake of many chemotherapeutic agents that are weak bases, thus further amplifying chemotherapy resistance. Therefore, it may be desirable for the stimulatory nutritional solution to contain sufficient amounts of basic constituents to overcome the local effects of increased lactate production.

In formulating the stimulatory nutritional solution, it may be desirous to add carbohydrates. Cancer cells rely heavily on glycolysis as a source of energy generation. See Buzby, G. P., Mullen, J. L., Stein, T. P., Miller, E. E., Hobbs, C. L., Rosato, E. F., (1980) Cancer, 45:1940-48; and Copeland, E. M., Mac Fadyen, B. V., Lanzotti, V. J., Dudrick, S. J., (February 1975) Am. J. Surg., 129:167-173. This reliance was verified when hepatoma cells were cultured in carbohydrate deficient media, leading to rapid adenosine triphosphate (ATP) loss and cell death. Ching, N., Grossi, C., Jham, G., Zurawinsky, H., Ching, C. Y., Nealon, Jr., T. F., (June 1984) Surgery, 95(6):730-8. In contrast, in the same study, normal cells in carbohydrate deficient media survived for much longer periods of time. In addition, sarcoma cells having a lower metabolism because of exposure to cold temperatures have been observed to have a glucose uptake 3-5 times lower than those at room temperature. Schwartz, G. F., Green, H. L., Bendon, M. L., Graham III, W. P., Blakemore, W. S., (February 1971) Am. J. Surg., 121:169-173.

Providing high carbohydrate loads to tumors should stimulate their metabolism and elevate the S phase population of cells. Unfortunately, this would also lead to worsening of the acidosis that is a major part of a tumor's abnormal microenvironment which renders tumor cells resistant to chemotherapy. See Moll, M., Vaupel, P. (eds.): Springer-Verlag Berlin Heidelberg, New York, 1998; and Buzby, G. P., Mullen, J. L., Stein, T. P., Miller, E. E., Hobbs, C. L., Rosato, E. F., (1980) Cancer, 45:1940-48. Therefore, only moderate carbohydrate loads should be included in the stimulatory nutritional solution, thus providing cells with adequate energy supply for replication but without over stimulating their metabolism. It may also be desirable to supplement the carbohydrates with sufficient bicarbonate or other basic constituents to partially offset the worsening acidosis that is an inevitable consequence of tumor hypermetabolism.

In the preferred embodiment of the invention, the first nutritional solution is depleted, either partially or wholly, of one or more of the above described nutritional components which are preferentially used by tumor cells. The second nutritional solution contains at least a conventional concentrations of the same components, with conventional concentrations being approximately equivalent to commercially available TPN solutions. The second nutritional solution may also be significantly enhanced with the nutritional components. The determination of which components are depleted in the first solution and which are added to the second solution is most effective when done on a case-by-case basis. The actual formulations are dependent upon the response of similar types of in vitro tumors when the process described below is performed.

In practicing the method of this invention, the results of treating in vitro tumors should inform the treatment of tumors in vivo to provide the most effective in vivo treatment. Tumor cells are therefore harvested from in vivo tumors via surgical resection or needle biopsy for treatment in vitro prior to any in vivo treatment being performed in accordance with this invention. Performing the method of this invention on different types of tumors in vitro is important to establish the baseline nutritional needs of the tumors without interference from the surrounding in vivo tissue and functions. The following outlines one possible process for performing this invention on in vitro tumors and describes how the information gained may be used in practicing this invention on in vivo tumors.

Cells obtained from the in vivo tumor are maintained in several mono-layer, long term cultures according to methods cited in published literature. See Izuishi, K., Kato, K., Ogura, T., kinoshita, T., Esumi, H., (2000) Cancer Res., 60(21):6201-7, the disclosure of which is incorporated herein by reference. A mono-layer culture is preferred over a spheroid culture because mono-layer cultures do not completely simulate some in vivo conditions which are undesirable during this phase of the process. For example, in spheroid cultures, but not in mono-layer cultures, tumor cells quickly generate a micro-environmental gradient that is similar to in vivo conditions. Such a microenvironment would lead to the formation of a heterogeneous group of cells with the different groups being characterized by decreasing growth activity from the viable rim to the dormant interior. Simulating such conditions in these cultures is undesirable because in this stage of the process, the aim is to determine cellular nutritional requirements under conditions of maximal growth. Such conditions are best represented in a mono-layer culture. At this stage, therefore, simulating the in vivo growth environment would be counter productive.

As indicated, tumor cells are obtained from biopsy or surgical sources and prepared in culture. The sample cells are prepared as 1×10⁶ cells plated in 25 cm² flasks. The cells are cultured in Dulbecco's modified essential medium (DMEM) supplemented with 10% fetal bovine serum (FBS), 100 units/ml of penicillin, 100 mcg/ml of streptomycin 2.5 mcg/ml of amphotericin B, and 2 mM of glutamine. To these mixtures varying concentrations of glucose, ranging from 2-20 mg/ml, are added. Multiple combinations of cultures are prepared and examined to determine which provide ideal growth conditions. The flasks are incubated at 37° C. in an environment of 5% carbon dioxide and 95% humidity. The culture media should be replaced every 72 hours, although the most rapid period of growth for most tumor cells is expected to be during the first 48 hours when the cell population is at its lowest levels. Growth quantification is evaluated using either cytometry or thymidine labeling as previously described herein.

Once the cultures have been established, nutrigrams are performed on fluids from the tumor cell cultures as the first step in determining the preferential nutritional components used by the tumor cells. An amino acid analysis is performed using an amino acid analyzer, such as the Hitachi L-8800. Lipid analysis is accomplished by gas-liquid chromatography using an instrument such as the Tracor 540 GC. Electrolyte and trace element analysis is performed using a standard clinical chemical analyzer such as the one made by Beckman. The nutrigrams of the tumor cell cultures are compared with normal cell nutrigrams.

Normal cell cultures are prepared and maintained in the same manner as the tumor cell cultures. Cells of different tissue origin including bone marrow and those of the tumor's tissue origin should be tested every time, as these are the normal cells most likely to be affected by nutritional manipulation of the tumor cells and the ensuing chemotherapy. The S phase fraction measurement of the cell cultures under these conditions and the supernatant nutrigrams are generated in the manner previously described in connection with the tumor cell cultures.

After obtaining nutrigrams from both the tumor cell cultures and the normal cell cultures, the data are analyzed, preferably using software. A spreadsheet program, such as Microsoft Excel, is sufficient for these purposes. Each amino acid, lipid, electrolyte, and trace metal concentration in the normal cell culture medium is subtracted from that in the cancer cell culture medium. The resulting values, hereinafter referred to as the “subtraction nutrigrams,” are expressed as percentages of the initial culture concentrations of the same components.

Once the subtraction nutrigrams are obtained, the information is analyzed to identify the nutrients which are preferentially used by the tumor cells. The largest negative and positive values in the subtraction nutrigrams are identified as the most important nutrients in the metabolism of the cancer cells. These nutrients are targeted in the subsequent formulation of the inhibitory and stimulatory nutritional solutions. As noted previously at least two general approaches may be employed, a first being a simple oversupply or depletion (partial or whole) of the targeted nutrients. For example, if arginine is found to be deficient in cancer cell cultures following rapid growth, then the inhibitory solutions will be devoid of arginine and the stimulatory solutions will have between a 2 to 100 times increase in the concentration of arginine. Alternatively, or in combination with the above approach, competing amino acids may be targeted. For example, lysine is an amino acid that competes for cellular uptake with arginine; thus the inhibitory solution should have enhanced concentrations of lysine, or approximately 2-100 times the lysine concentration in commercially available TPN solutions, whereas the stimulatory solution should be deficient in lysine.

In formulating the nutritional solutions, for any type of tumor which has not previously been treated using this method, the initial in vitro process should include use of a relatively wide range of concentration variations. However, where a type of tumor has been treated with this method, it is acceptable to use a narrow range of concentration variations. For the treatment of most tumors, it is likely that an increase of between 5 to 10 times the normal concentration of each component will be sufficient to produce optimal results.

Following the formulation of the various test inhibitory and stimulatory nutritional solutions, the solutions are used to nourish cultures of tumor cells and normal cells identical to the ones previously prepared. The starting cell concentrations and the cell media are prepared as before, with the exception that the cells are plated in solutions containing 75% by volume of the above stated culture media and 25% by volume of the inhibitory or stimulatory solutions. This proportion is chosen because the average circulating volume of blood for a 70 Kg individual is 5-6 liters while the daily volume of TPN that would be infused into such a patient is in range of 1-1.5 liters per day. Alternatively, in order to better simulate in vivo conditions, cultures may be started in smaller initial volumes of the original culture media, with the inhibitory or stimulatory solutions gradually added to the culture media over time. The culture media should be changed daily, with cellular growth and the S phase portions being measured at periodic intervals. The most promising combinations of inhibitory and stimulatory solutions may therefore be identified by plotting growth curves of the cell cultures over time.

The inhibitory solutions ideal for use in combination with the stimulatory solutions should produce relatively flat growth in the normal cells and the growth curve of the tumor cells should flatten after showing growth for a short period of time. The flattening of the growth curve is indicative of cell phase arrest in the tumor cells due to a lack of proper nutrients needed for replication. Conversely, the stimulatory solution(s) ideal for use in combination with the inhibitory solutions should produce relatively flat growth in the normal cells, while the tumor cells should show significant growth. In addition, the periodic cytometry should indicate a particular time period in which the population of tumor cells in the S phase is elevated.

After specific solutions are identified as ideal for use in combination, cells are again cultured as before to further verify which specific combinations work best. However, this time all cultures start with a culture medium that is 75% by volume of DMEM with 10% FCS, and 25% by volume of the inhibitory solution. Following the period of time, as determined by the previous step, in which growth becomes arrested, the cells are transferred to a medium containing 25% by volume of the stimulatory solution and 75% by volume of DMEM supplemented with 10% FCS. The cells are incubated on this medium for the period of time, as determined by the previous step, for which it took to have the most cells in the S phase. The S phase portion of the tumor cells is verified at the end of each cycle. Each cycle is defined as the period of time during which the cells are exposed to the inhibitory solution once and to the stimulatory solution once. Cycles are repeated as necessary until the portion of cells synchronously in the S phase is elevated, and preferably until 90 to 100% of the cells are synchronously in the S phase, over any 4 hour period. Alternatively cultures may also be carried out in gradually increasing portions of TPN up to 100% of TIPN/TSPN supplemented with 10% FCS to more accurately simulate the in vivo conditions.

In addition to the above process for determining which nutrients should be used to formulate the nutritional solutions for inducing S phase synchronization, other stimuli may also be introduced to the cultures to further enhance or accelerate S phase synchronization. Such stimuli may include anti-hormone therapy during administration of the inhibitory solution and hormone therapy during the administration of the stimulatory solution for hormone sensitive tumors; other stimuli which cause phase specific cell cycle arrest such as quinidine, hydroxyurea, lovastatin and glucocorticoids; drugs such as isoproterenol which have some efficacy in recruiting dormant cells into the cell cycle; or chemosensitizers such as verapamil can be combined with the stimulatory solution immediately prior to the infusion of chemotherapy to augment its effects. However, use of many of these other stimuli in vitro should be carefully weighed, as the overall goal of the in vitro process is to derive viable nutritional solutions which can induce S phase synchronization for the in vivo process, and many of these additional stimuli have undesirable side effects at pharmacological doses, thereby limiting their usefulness in vivo.

Once the process is completed on a particular type of tumor in vitro, general guidelines about the timing and optimal culture settings can be established. Additionally the control nutrigrams under different conditions will have been established and will not need to be repeated for different in vivo environments every time, i.e., for different patients. The information learned from the treatment of in vitro tumors should be applied to the treatment of in vivo tumors. In treating patients with cancer, two TPN solutions are prepared for the patient. The first TPN solution is formulated from the inhibitory solution determined to be most effective in the in vitro setting. Likewise, the second TPN solution is formulated from the stimulatory solution determined to be most effective in the in vitro setting.

Once TPN solutions are prepared, they may be given to a patient as outlined above in an alternating manner, starting with the inhibitory solution. The S phase synchronization of the tumor cells is monitored by the PET scanning process discussed previously. Following a determination that the population of tumor cells that are synchronously in the S phase is elevated, chemotherapy is administered. During administration of the solutions and the chemotherapy, a patient's oral intake should be restricted to small amounts of water only. By restricting the oral nutritional intake of the patient, the nutrient variability introduced by the patient's diet may be eliminated.

The side effects a patient suffers because of the above described nutritional manipulation process should be negligible. As previously stated, most normal cells have periods of dormancy in between replications. Normal cells are thus have lower nutritional requirements than cancer cells. In addition, normal cells have cellular synthetic machinery which may be used to produce many non-essential amino acids and therefore are not vulnerable to plasma depletion of those amino acids. Finally, the growth cycle of normal cells is controlled by a complex chain of biochemical regulatory signals. Thus, they are not likely to go in and out of the replication part of the cell cycle because of the depletion or overabundance of plasma nutrient levels.

Thus a method for increasing the efficacy of chemotherapy in the treatment of cancer has been disclosed. While embodiments of this invention have been shown and described, it would be apparent to those skilled in the art that many more modifications are possible without departing from the inventive concepts herein. The invention, therefore, is not to be restricted except in the spirit of the following claims.

What is claimed is:

1. A method of treating a tumor comprising:
alternately providing to the tumor a first nutritional solution and a second nutritional solution while depriving the tumor of other external sources of nutrition, wherein the first nutritional solution is formulated to inhibit tumor growth and the second nutritional solution is formulated to stimulate tumor growth;
determining when a population of tumor cells that are synchronously in the S phase of the cell cycle is elevated; and
treating the tumor with chemotherapy while the population of tumor cells that are synchronously in the S phase is elevated.

2. The method according to claim 1 further comprising identifying nutritional components preferentially used by the tumor and formulating the first and second nutritional solutions based on the nutritional components.

3. The method according to claim 2, wherein the nutritional components comprise nutrients selected from the following groups: amino acids, lipids, electrolytes, and trace metals.

4. The method according to claim 2, wherein the first nutritional solution is depleted, either partially or wholly, of the nutritional components.

5. The method according to claim 2, wherein the first nutritional solution is enhanced with nutrients which compete for cellular uptake with the nutritional components.

6. The method according to claim 2, wherein the second nutritional solution is enhanced with one or more of the nutritional components.

7. The method according to claim 2, wherein the second nutritional solution is depleted, either partially or wholly, of nutrients which compete for cellular uptake with the nutritional components.

8. The method according to claim 2 further comprising enhancing the second solution with carbohydrates.

9. The method according to claim 1, wherein determining whether there has been an elevation in the population of tumor cells that are synchronously in the S phase includes performing a thymidine labeling analysis of the tumor.

10. The method according to claim 1, wherein the first and second nutritional solutions are provided to the tumor through total parenteral nutrition (TPN).

11. The method according to claim 1, wherein determining whether there has been an elevation in the population of tumor cells that are synchronously in the S phase includes measuring cellular absorption of a radioactive labeled molecule.

12. A method of treating a tumor comprising:
identifying nutritional components that are preferentially used by the tumor;
formulating a first nutritional solution and a second nutritional solution based on the nutritional components used preferentially by the tumor, wherein the first nutritional solution is formulated to inhibit tumor growth and the second nutritional solution is formulated to stimulate tumor growth;
alternately providing to the tumor the first nutritional solution and the second nutritional solution while depriving the tumor of other external sources of nutrition;
determining when a population of tumor cells that are synchronously in the S phase of the cell cycle is elevated; and
treating the tumor using chemotherapy while the population of tumor cells that are synchronously in the S phase of the cell cycle is elevated.

13. The method according to claim 12, wherein the nutritional components comprise nutrients selected from the following groups: amino acids, lipids, electrolytes, and trace metals.

14. The method according to claim 12, wherein the first nutritional solution is depleted, either partially or wholly, of one or more of the nutritional components.

15. The method according to claim 12, wherein the first nutritional solution is enhanced with nutrients which compete for cellular uptake with the nutritional components.

16. The method according to claim 12, wherein the second nutritional solution is enhanced with one or more of the nutritional components.

17. The method according to claim 12, wherein the second nutritional solution is depleted, either partially or wholly, of nutrients which compete for cellular uptake with the nutritional components.

18. The method according to claim 12 further comprising enhancing the second solution with carbohydrates.

19. The method according to claim 12, wherein identifying whether there has been an elevation in the population of tumor cells that are synchronously in the S phase includes performing a thymidine labeling analysis of the tumor.

20. The method according to claim 12, wherein the first and second nutritional solutions are provided to the tumor through total parenteral nutrition (TPN).

21. The method according to claim 12, wherein identifying whether there has been an elevation in the population of tumor cells that are synchronously in the S phase includes measuring cellular absorption of a radioactive labeled molecule.
